(19) United States
(12) Patent Application Publication
(10) Pub. No.: US 2024/0050150 A1
(43) Pub. Date: Feb. 15, 2024

(54) CARDIAC ABLATION CATHETER AND SYSTEM THEREOF

(71) Applicant: CathRx Ltd, Homebush Bay, NSW (AU)

(72) Inventors: Milanjot Singh Assi, Kellyville Ridge, (AU); Pengjin Pan, Sydney (AU); lan KimSeng Fong, Lane Cove, NSW (AU); Daniel Meckes, Springwood, NSW (AU)

(21) Appl. No.: 18/257,760

(22) PCT Filed: Dec. 21, 2021
(86) PCT No.: PCT/IB2021/000894

§ 371 Date: Jun. 15, 2023

Related U.S. Application Data
(60) Provisional application No. 63/129,699, filed on Dec. 23, 2020.

(51) Int. Cl. A61B 18/14

(52) U.S. Cl. CPC A61B 18/1492; A61B 2018/1407

(57) ABSTRACT

A pulsed field ablation catheter for use in atrial fibrillation treatment having a distal steerable variable loop with multiple electrodes coupled to the distal end of a multilumen shaft and a handle coupled to the proximal end of the multilumen shaft. A shape imparting mechanism coupled to the handle is configured to change the shape of steerable loop by manipulating a steering mechanism to steer the steerable loop to the treatment site and a rotational actuator to change the diameter of the variable loop to better interface with varying human anatomy.

### CROSS-REFERENCE TO RELATED APPLICATIONS

This application is a national phase entry under 35 U.S.C. § 371 of International Patent Application PCT/IB2021/000894, filed Dec. 21, 2021, designating the United States of America and published as International Patent Publication WO 2022/136918 A1 on Jun. 30, 2022, which claims the benefit under Article 8 of the Patent Cooperation Treaty to U.S. Patent Application Ser. No. 63/129,699, filed Dec. 23, 2020.

### TECHNICAL FIELD

The present disclosure relates to a steerable cardiac catheter and, more particularly, to an intracardiac ablation catheter, systems and improvements thereto.

### BACKGROUND

Atrial fibrillation (AF) is the most common heart condition throughout the world. In the United States, approximately 410,000 hospitalizations, 5 million office visits, and 3,800,000 emergency department visits occur annually. It is estimated that approximately 67% of patients seen in the emergency department presenting with AF require hospital admission and 35% of all hospital admissions for arrhythmia are attributable to AF. Among the patients who develop AF, pharmacological approaches are often necessary, but unfortunately ineffective. When medications lose their effectiveness, surgery and/or catheter-based device therapy is required. To date, surgery remains an invasive alternative not suitable for all patients and current device therapies are thermally unselective by nature causing potential life-threatening complications. To address these problems, investigation of treatments to reduce therapy related complications and reduce the sizable burden of the disease are needed.

Research shows that pulsed field ablation (PFA), also known as irreversible electroporation, is a highly effective catheter-based cardiac ablation therapy that potentially improves AF outcomes (e.g., Reduction in AE, reductions in reoccurrence, improved QoL). When compared to conventional radiofrequency ablation (RFA) or cryo-therapeutic cardiac ablation devices, PFA therapy is non-thermal and tissue selective. This modality reduces the risk of cardiac tamponade, stroke, esophageal injury, pulmonary vein stenosis, and phrenic nerve injury associated with previous generation cardiac ablation therapies. While current care plans deal with early diagnosis and pharmacological treatments to mitigate the risks associated with surgery and catheter-based device therapies, PFA eliminates those risks entirely.

In 2018, a study entitled “Electroporation and its relevance for cardiac catheter ablation” stated that “Total applied current, not delivered power (watts), energy (joules), or voltage, is the parameter that most directly relates to the local voltage gradient that causes electroporation.” It was also found that irreversible myocytic destruction is best achieved through bipolar monophasic and bipolar biphasic waveforms, with bipolar biphasic demonstrating the greatest cellular response. It was also reported that asymmetrical waveforms may have an even greater impact on myocardium leading to deeper lesions for a more efficacious PFA treatment. Nevertheless, each therapeutic waveform is rapidly delivered in microseconds versus minutes as needed for conventional RF and cryotherapies. This speed reduces the overall procedure time while also limiting resistive or joule heating of surrounding anatomy making PFA technology safer and more efficacious overall.

To harness the therapeutic potential of PFA the dominant technologies have focused on pulmonary vein isolation technologies including loops, baskets, and balloons. Although these catheter designs target the pulmonary veins, where approximately ninety-four percent of chaotic electrical activity occurs, they are limited by their ability to adapt to the changing intracardiac landscape including the ability to create therapeutic lesions inside the pulmonary veins as well as on the posterior wall of the left atrium. Achieving adequate lesion formation in these regions is not trivial. Pulmonary vein isolation requires lesions of a few (>2) millimeters deep to ensure transmural ablation of the usually thin myocardial sleeves. The posterior wall may require lesions greater than 3 mm in depth. In both cases, a sturdy and steerable catheter having varying catheter sizes and shapes is ideal to interface with the various cardiac anatomies in need of treatment.

Published Patent Cooperation Treaty Patent Application No. WO2016197186 describes a cardiac ablation double loop catheter. The described double loop catheter is adapted for delivering ablation energy, the catheter comprising a catheter sheath having a proximal end, a distal end and at least one lumen extending therethrough and having a first loop structure having a proximal end, and a distal end and a second loop structure having a proximal end and a distal end. The first loop structure and the second loop structure being receivable in the at least one lumen of the catheter sheath, and the first loop structure and the second loop structure being configurable to extend distally of the distal end of the catheter sheath. The first loop structure comprising a first electrode near to the distal end of the first loop structure and the second loop structure comprising a second electrode near to the distal end of the second loop structure, wherein the first loop structure and the second loop structure are displaceable relative to each other. The configuration of this catheter could be improved with better sealing and electrical isolation. Also, the ablation electrodes may overlap when the loop size of the distal end is minimized, which is generally undesirable for surgery.

Patent Cooperation Treaty Patent Application No. PCT/IB2020/056538 describes an improved cardiac ablation catheter wherein the catheter is limited to a linear or J-curve shaped catheter. The present disclosure relates to various improvements over the prior art design described within '538.

It would be advantageous to provide a PFA catheter design that could create a variety of circular loop diameters to allow for greater flexibility and efficiency of use during medical procedures.

It would be advantageous to provide a PFA catheter design having the ability to act as a vehicle for interventional or diagnostic tools or devices.

It would be advantageous to have a device that further mitigates the risk of thermal ablation cardiac therapies and devices while also improving the ease of use and efficacy.

Any discussion of prior art throughout the specification should in no way be considered as an admission that such prior art is widely known or forms part of common general knowledge in the field.

### BRIEF SUMMARY

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

It would be advantageous to provide a PFA variable loop catheter capable of creating different lesion shapes and sizes without the crossing of electrodes that may lead to electric surging and or arcing.

It would be advantageous to provide a PFA catheter capable of being a vehicle for interventional or diagnostic tools or devices simultaneously/concurrently during the percutaneous procedure.

It would be advantageous to provide a multi-device catheter with a decreased risk of thrombus formation and stroke.

It would be advantageous to provide a PFA catheter having a delivery lumen of low-flow low-pressure to eliminate the need for costly pumps and fluid flow devices.

It is an object of the present disclosure to overcome or ameliorate at least one of the disadvantages of the prior art, or to provide a useful alternative.

A first aspect of the present disclosure may provide a cardiac ablation variable catheter comprising:
i. a shaft having a proximal end, a distal end, and at least one insulative lumen and at least one delivery lumen;
ii. a first steerable loop capable of being inserted into the insulative lumen and a second steerable loop capable of being inserted into the delivery lumen and wherein the delivery lumen is adapted to mounted within the insulative lumen;
iii. a handle coupled to the proximal end of the shaft, the handle having a steering mechanism;
iv. a shape imparting element having a proximal end and a distal end; the proximal end is adapted to be coupled to the steering mechanism and the distal end of the element is adapted to be coupled to the steerable loop and wherein the shape imparting element is positioned within the insulative lumen; and
v. a plurality of electrodes positioned on the steerable loop electrically coupled to at least one electrical connector, the at least one electrical connector being configured to electrically coupled with an electrical pulsed field ablation energy source to power the plurality of electrodes for treatment.

Further, wherein the cardiac ablation variable catheter comprises the shaft having a first diameter, a proximal end and distal end wherein the distal end is joined to distal loop section having a second diameter and wherein the join is achieved by the mating of a tongue and groove positioned on corresponding mating portions of the distal loop section and distal end.

Further, wherein the catheter includes a plurality of lumens.

Further, wherein a plurality of electrodes positioned on the second steerable loop, the electrodes are configured to be electrically coupled with an electrical pulsed field ablation energy source to power the plurality of electrodes for treatment; and wherein the plurality of electrodes are spaced at predetermined intervals on the arc of the second half of the circle.

Further, wherein the steering loop is reduced to the minimum circle, the plurality of electrodes is positioned apart around the circumference of the circle.

Further, wherein the predetermined intervals are equidistant.

Further, wherein the plurality of electrodes is 12 electrodes.

Further, wherein the electrodes comprise alternative positive and negative electrodes.

Further, wherein adjacent pairs of electrodes fire simultaneously.

Further, wherein localized first pairs of similar electrodes fire simultaneously, with corresponding localized second pairs of opposite electrodes firing subsequent to the first pairs.

Further, wherein the steerable loop in a between the maximum and minimum diameters has no electrode overlap other electrodes on the steerable loop.

Further, wherein a non-metallic braid is encapsulated within the wall of the composite tube proximal to the distal end of the shaft.

Further, wherein the braid is constructed of a non-electrically conductive material.

In another aspect there is provided, a cardiac ablation variable catheter comprising:
i. a shaft having a proximal end, a distal end, and at least one insulative lumen;
ii. a first steerable loop capable of being inserted into the insulative lumen;
iii. a handle coupled to the proximal end of the shaft, the handle having a steering mechanism;
iv. a shape imparting element having a proximal end and a distal end; the proximal end is adapted to be coupled to the steering mechanism and the distal end of the element is adapted to be coupled to the steerable loop and wherein the shape imparting element is positioned within the insulative lumen; and
v. a plurality of electrodes positioned on the steerable loop electrically coupled to at least one electrical connector, the at least one electrical connector being configured to electrically coupled with an electrical pulsed field ablation energy source to power the plurality of electrodes for treatment;
vi. wherein a plurality of electrodes positioned on the first steerable loop, the electrodes are configured to be electrically coupled with an electrical pulsed field ablation energy source to power the plurality of electrodes for treatment; and wherein the plurality of electrodes are spaced at predetermined intervals on the arc of the second half of the circle.

Further, wherein the shaft having a first diameter, a proximal end and distal end wherein the distal end is joined to distal loop section having a second diameter and wherein the join is achieved by the mating of a tongue and groove positioned on corresponding mating portions of the distal loop section and distal end.

Further, wherein the steering loop is reduced to the minimum circle, the plurality of electrodes is positioned around the circumference of the circle.

Further, wherein the predetermined intervals are equidistant. Further, wherein the plurality of electrodes is 12 electrodes.

Further, wherein the steerable loop in a between the maximum and minimum diameters has no electrode overlap other electrodes on the steerable loop.

Further, wherein a non-metallic braid is encapsulated within the wall of the composite tube proximal to the distal end of the shaft.

Further, wherein the braid is constructed of a non-electrically conductive material.

### BRIEF DESCRIPTION OF THE DRAWINGS

The accompanying drawings, which are incorporated herein and constitute part of this specification, illustrate the presently preferred embodiments of the present disclosure, and, together with the general description given above and the detailed description given below, serve to explain the features of the present disclosure. In the drawings:

FIG. 1 depicts a perspective view of a first preferred embodiment of a pulsed field ablation catheter;

FIG. 2 depicts a perspective view of the first preferred embodiment of a pulsed field ablation catheter and variable loop;

FIGS. 3A and 3B depict a cross-sectional view and perspective view of the device of FIG. 1;

FIG. 4 depicts a perspective assembly view of a second embodiment of the present disclosure featuring a single loop configuration;

FIGS. 5A and 5B depict an assembly view of the pulsed field ablation catheter's handle of FIG. 4 including its internal mechanical parts and mechanisms;

FIGS. 6A and 6B depict an assembly and perspective view of the shape imparting steering and loop sizing mechanism of the second embodiment;

FIG. 7 depicts a perspective exploded and enlarged view of the pulsed field ablation catheter's combined butt-joint and tongue-and-groove joint of the second embodiment;

FIGS. 8A, 8B and 8C depict alternative configurations and variants of the tongue 33 of the second embodiment;

FIGS. 9A and 9B depict a profile and side view of a preferred design of the combined butt-joint and tongue-and-groove joint of the second embodiment;

FIG. 10A depicts a third preferred embodiment of the catheter featuring a distal variable loop illustrating a large diameter loop and a small diameter wherein the high-voltage delivering electrodes do not cross one another during loop sizing;

FIG. 10B depicts the catheter of FIG. 10A, with the loop diameter decreased by one half;

FIG. 11 depicts a fourth embodiment of the present disclosure featuring a cardiac ablation catheter with a single variable loop distal end in a compacted configuration;

FIG. 12A depicts a distal end portion of an ablation catheter of the present disclosure;

FIG. 12B depicts the distal tip portion of the catheter of FIG. 12A, with a pull wire shown;

FIG. 12C depicts the distal tip portion of the catheter of FIG. 12A, with a loop wire extending distally therefrom;

FIG. 12D depicts an exemplary method for attaching the pull wire to the distal tip of the catheter;

FIG. 13 depicts the embodiment of FIG. 11 in an expanded configuration;

FIG. 14 depicts an enlarged cut away view of a portion of the embodiment of FIG. 11;

FIG. 15 depicts a top view of the embodiment of FIG. 11;

FIG. 16 depicts a top view of the embodiment of FIG. 11;

FIG. 17 depicts a top view of embodiment in FIG. 11 in a compact configuration; and

FIG. 18 depicts a top view of a further variant of the embodiment of FIG. 11.

### DETAILED DESCRIPTION

In the drawings, like numerals indicate like elements throughout. Certain terminology is used herein for convenience only and is not to be taken as a limitation on the present disclosure. The terminology includes the words specifically mentioned, derivatives thereof and words of similar import. The embodiments illustrated below are not intended to be exhaustive or to limit the present disclosure to the precise form disclosed. These embodiments are chosen and described to best explain the principle of the present disclosure and its application and practical use and to enable others skilled in the art to best utilize the present disclosure.

Reference herein to “one embodiment” or “an embodiment” means that a particular feature, structure, or characteristic described in connection with the embodiment can be included in at least one embodiment of the present disclosure. The appearances of the phrase “in one embodiment” in various places in the specification are not necessarily all referring to the same embodiment, nor are separate or alternative embodiments necessarily mutually exclusive of other embodiments. The same applies to the term “implementation.”

As used in this disclosure, the word “exemplary” is used herein to mean serving as an example, instance, or illustration. Any aspect or design described herein as “exemplary” is not necessarily to be construed as preferred or advantageous over other aspects or designs. Rather, use of the word exemplary is intended to present concepts in a concrete fashion.

The word “about” is used herein to include a value of +/−10 percent of the numerical value modified by the word “about” and the word “generally” is used herein to mean “without regard to particulars or exceptions.”

Additionally, the term “or” is intended to mean an inclusive “or” rather than an exclusive “or.” That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in this disclosure and the appended claims should generally be construed to mean “one or more” unless specified otherwise or clear from context to be directed to a singular form.

Unless explicitly stated otherwise, each numerical value and range should be interpreted as being approximate as if the word “about” or “approximately” preceded the value of the value or range.

Preferred embodiments of the present disclosure will now be described with reference to the accompanying drawings and non-limiting examples.

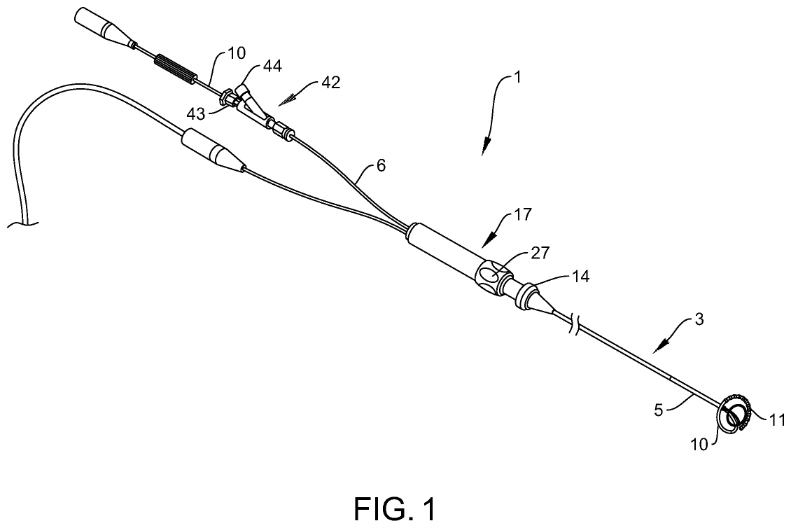

FIG. 1 depicts a perspective view of a first preferred embodiment of the present disclosure in the form of device or system including a pulsed field ablation catheter 1 for use in atrial fibrillation treatment. The ablation catheter 1 includes a distal steerable variable loop 2 coupled to the distal end of a multilumen shaft 3, and a handle 17 coupled to the proximal end of the multilumen shaft 3. Within the ablation catheter 1 is a shape imparting mechanism that is configured to change the shape of steerable loop 2 by manipulating a steering mechanism 14 to steer the steerable loop to the treatment site, and a rotational actuator 27 configured to change the diameter of the variable loop 2 to better interface with varying human anatomy. Please note that in this specification, variable loop and steerable loop are the same or similar mechanisms.

An electrical connector 40 is provided to connect the ablation catheter 1 to an energy source (not shown) to provide electrical pulsed field ablation energy to a plurality of electrodes 11 arranged on a distal portion of the ablation catheter 1. The energy source (not shown) is typically a waveform generator capable of generating electrical currents to induce the ablation therapy when connected to the first preferred embodiment of the present disclosure.

The ablation catheter 1 includes a multilumen catheter, wherein the catheter is adapted to be an ablation catheter with a first loop 101 and a second loop 100, wherein the first loop 101 is adapted to be nested inside the lumen of the second loop 100. Both first and second loops may move independent of each other in terms of shape, sizing and positional movement.

The ablation catheter 1 also includes a Y-connector 42 or bifurcated connector coupled to a delivery lumen 6 having a port hub 43 configured to receive interventional or diagnostic tools or devices 10 to deliver to the treatment area. The interventional or diagnostic tools or devices 10 may be used simultaneously/concurrently during a percutaneous procedure. Many types of interventional or diagnostic tools or devices 10 may be introduced into the heart through the ablation catheter 1 including: ECG sensing and pacing catheters, biopsy forceps, imaging devices, and ablation catheters. The Y-connector also includes a fluid inlet 44 that may be used as a continuous flow path for fluid, such as saline, into the treatment area to decrease the risk of thrombus formation and stroke. The fluid inside the delivery lumen also acts as a friction reduction mechanism allowing the interventional or diagnostic tools or devices 10 to pass through the lumen more freely. Biocompatible fluid or fluid may also be introduced or excreted by the delivery lumen 6 wherein transfusion blood or fluid is slowly excreted from the delivery lumen 6. This has the advantage of preventing or limiting the risk of blood clots or thrombus forming at the exit port positioned within the patient, when is use. The constant slow flow of fluid from the delivery lumen 6 washes the distal end of the catheter, preventing thrombus where the diagnostic tool exits the shaft 3.

In a further variant, the delivery lumen may include an aperture of smaller diameter to the delivery lumen at the distal end of the shaft that is adapted to impede or reduce the flow rate of the fluid.

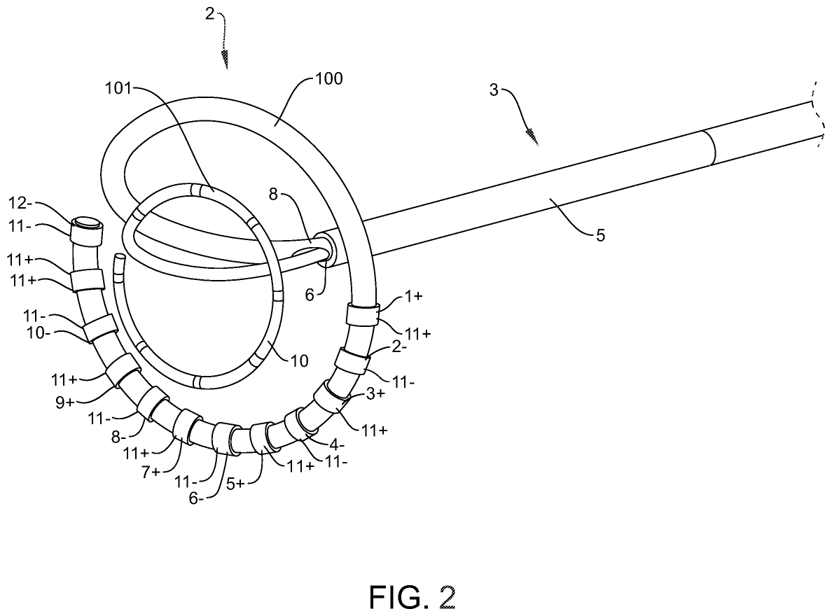

FIG. 2 depicts a perspective view of the distal end of the pulsed field ablation catheter 1 showing the steerable loop 2 with a plurality of ablation electrodes 11 of the first preferred embodiment. The variable loop 2 is coupled to an insulative lumen 8 at the distal end of the multilumen shaft 3. The distal end of the multilumen shaft 3 should also be flexible or steerable, so the multilumen shaft 3 is designed with a flexible region or deflection zone 5. The plurality of electrodes 11 are electrically coupled to the energy source 41. The steerable loop 2 has a smaller diameter than the multilumen shaft 3 so that it does not interfere with the delivery lumen 6. FIG. 2 shows an interventional or diagnostic tool or device 10 extending from the delivery lumen 6.

FIG. 2 depicts a configuration with twelve ablation electrodes 11 mounted or positioned on the outer surface of the distal end of the variable loop ablation catheter. Electrodes 11 can be alternating positive and negative electrodes 11+, 11−, respectively. In a first embodiment, adjacent pairs of electrodes 11+, 11− can fire simultaneously. In an alternative embodiment, localized pairs of similar electrodes can fire simultaneously, with the corresponding localized pairs of opposite electrodes firing subsequent to the first pair. By way of example, if electrodes 11+ are sequentially numbered as 1+, 3+, 5+, 7+, 9+, 11+ and negative electrodes 11− are sequentially numbered 2−, 4−, 6−, 8−, 10−, 12−, it has been found that a firing sequence such as 1+/3+ and then 2−/4− delivers a higher ablation energy without sparking.

The diagnostic tool 10 also includes a second set of electrodes 45, wherein the purpose and function of the second set of the electrodes 45 is to detect atrial activity, when in use. The second set of electrodes 45 may be used to detect and map the electrical activity of the heart when linked to mapped software. The second set of electrodes 45 includes eight electrodes mounted or bonded to the outer surface of the diagnostic tool 10. When in use, the diagnostic tool 10 may be moved and deflected independent of the variable ablation loop 2.

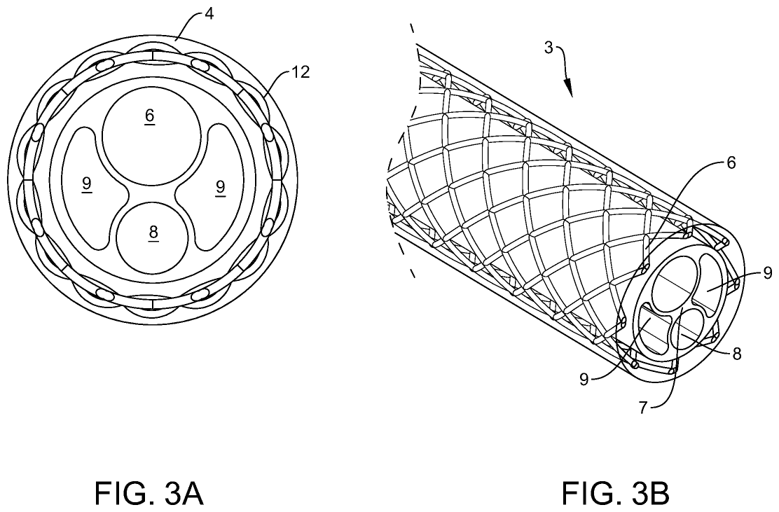

FIGS. 3A and 3B depict a cross-sectional view profile of the first preferred embodiment shown in FIG. 1 and perspective view of the multilumen composite catheter shaft 3 and internal lumens. In the embodiment shown, the multilumen composite catheter shaft 3 is a composite tube 4 having a braid fiber 12 positioned at an angle to maximize flexibility and torsional resistance. The braid fiber 12 may be non-conductive or conductive braid fiber. The multilumen composite catheter shaft 3 includes the delivery lumen 6, the septum 7, the insulative lumen 8, and two eccentric lumens 9. The septum 7 allows the delivery lumen 6 and the insulative lumen 8 to be separated while maintaining adequate wall thickness so that a bifurcation can occur in the proximal handle and in the distal shaft. This unique feature allows the delivery lumen 6 to operate without leakage of fluid. It also permits adequate insulation of a shape-imparting mechanism 13 throughout the insulative lumen 8. Additionally, the multilumen composite catheter shaft 3 provides the first and second eccentric lumens 9 through which multiple high-voltage wires can pass unhindered by the shape imparting nitinol mechanism and the delivery lumen 6. Some of the wires may be used to power a plurality of electrodes 11. The eccentric lumens 9 have a generally kidney-shaped cross-sectional profile as depicted in FIG. 3A and this shape aids in fitting wiring (not shown in FIGS. 3A and B) within the eccentric lumens 9.

Preferably, the braid fiber 12 is embedded and encapsulated within the outer wall of the composite tube 4. The braid fiber 12 is sealed within the wall and may allow for additionally reinforcement of the tube 4 to prevent breakage during bending or flexing. The braid fiber 12 is generally in form a woven mesh with a predetermined pore size of between a range of 0.1 mm to 2 mm. Preferably, the braid fiber 12 may be constructed of a non-metallic material to prevent or limit noise effects on wiring and sensors within the device. Preferably, the braid fiber may be constructed of a reinforced nylon, polyurethane, or Kevlar material. Kevlar is described in U.S. Pat. No. 3,819,587.

The multilumen composite catheter shaft 3 may preferably also be steerable. This requires the composite tube 4 to have a flexible region or deflection zone 5 at the distal end. The mechanical performance of the deflection zone 5 can demonstrate improved torque without compromising flexibility by replacing the non-conductive braid fiber with a conductive yet relatively more rigid metal fiber. This is because metals are inherently stiffer than insulators and therefore contribute a greater mechanical resistance to deformation. However, precise arrangement of the metal braid fibers is required to maximize flexibility but also improve torsional resistance. The deflection zone 5 is preferably reinforced by the braid fiber 12.

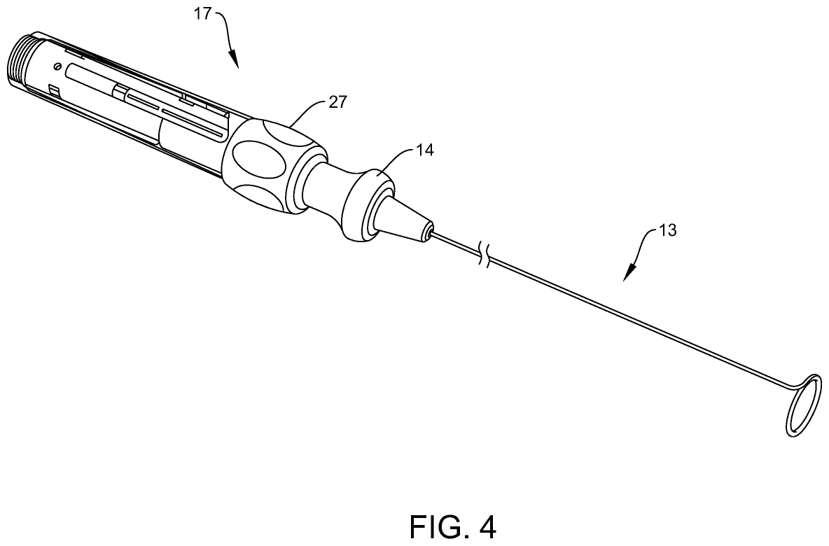

FIG. 4 depicts the catheter's handle 17 of second preferred embodiment of the present disclosure having the shape-imparting steering mechanism 13 and a rotation actuator 27. The steerability of the pulsed field ablation catheter 1 may include a shape-imparting mechanism 13 running from an actuator or steering mechanism 14 through the pulsed field ablation catheter 1 to the distal end. The shape-imparting mechanism 13 may be a mechanical actuator moving an internal pull-tube 15, or an electrical actuator that applies an electrical current to change the shape of a shape-memory tube. Movement of the steering mechanism 14 changes the shape of the shape imparting mechanism to steer the steerable loop 2 to the treatment site. Once at the treatment site, movement of the rotational actuator 27 is configured to change the diameter of the variable loop 2 to better interface with varying human anatomy. The second preferred embodiment is similar to the first preferred embodiment except wherein the diagnostic tool 10 has been removed from this configuration.

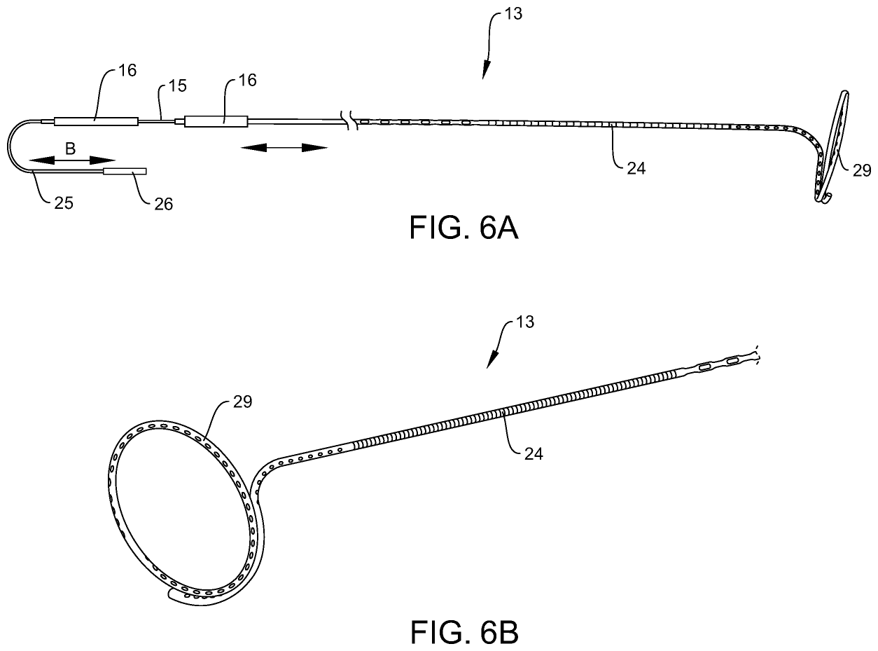

FIGS. 5A and 5B depict internal views of the pulsed field ablation catheter's handle depicted in FIG. 4 and actuator mechanisms. The mechanical actuator includes two larger metal rods 16 that terminate at the distal most ends of the shape-imparting mechanism 13 and shape-memory tube. The metal rods 16 are key fitted or glued into the catheter's handle 17. The catheter's handle comprises an actuator 14 or plunger, an external shell 18 and an inner core 19, a rotator 20 and linearly displacing pull-mechanism 21. The inner core 19 being secured to the external shell 18 by way of a key feature 22 and a proximal handle plug 23 that keys the external shell 18 with the inner core 19 by a thread and teeth. The actuator 14 being concentrically mated with the inner core 19 where linear displacement is limited by at least one shape imparting mechanism metal rod key feature. When the actuator 14 is displaced linearly A, the pull-tube 15 remains fixed to the inner core 19 and external shell 20 whereas the actuator 14 or plunger as well as the shape-imparting mechanism 13 are displaced or pulled away from one another causing a reduced stiffness skive 24 to buckle further deflecting the catheter's deflection zone 5.

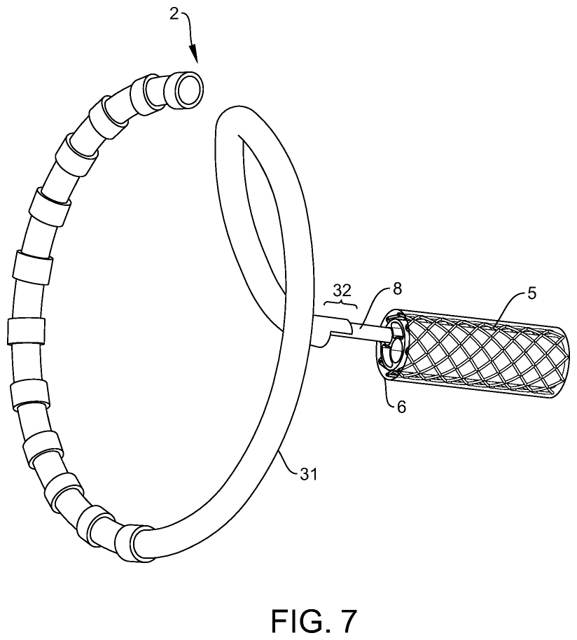

FIGS. 6A and 6B further illustrate the shape imparting and loop sizing mechanisms of the second preferred embodiment. The variable loop sizing mechanism of the pulsed field ablation catheter 1 includes a pull-wire 25 that runs inside the pull-tube 15 and shape-imparting mechanism 13. The pull-wire 25 also includes a larger rod 26 terminating at its distal most end. The larger rod 26 is also press-fitted or glued into a slot located in the linearly displacing pull-mechanism 21. As discussed above, when the actuator is moved linearly A, the pull-tube 15 remains fixed and the shape-imparting mechanism 13 moves linearly A. The pull-mechanism 21 is linearly displaced B by the rotator 20. As the rotator 20 is turned by rotation actuator 27, the rotator 20 transmits rotation energy into linear displacement B by sliding the pull-mechanism 21 along a cork-screw path 28 until it terminates its path 29 at the distal end of the inner core 19. This mechanical motion causes the variable loop 2 to change its diameter due to the pull-wire 25 actuating the reduced stiffness shape-imparting mechanism's distal loop 29.

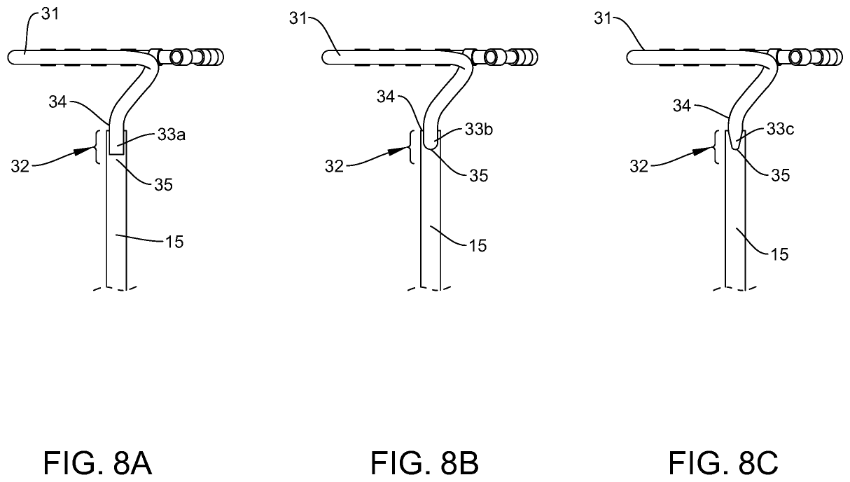

FIG. 7 depicts a perspective exploded view of the pulsed field ablation catheter 1 with a combined butt-joint and tongue-and-groove joint 32. In the embodiment shown, an insulative lumen 8 extends distally from the multilumen composite catheter shaft 3 and comprises an inner radius and an inner lumen coupled to the deflection zone 5. The insulative lumen 8 extends from the multilumen catheter shaft 3 and deflection zone 5 through a distal loop section 31 having a smaller outer diameter than the deflection zone 5. The distal loop section 31 is made of a non-conductive material having a low flexural modulus to further facilitate the work of an internal variable loop nitinol shape-imparting mechanism 13. The non-conductive composite tube 15 balances a low flexural modulus, a high ultimate tensile strength, and optimized inner radii to allow for the nitinol shape imparting mechanism to mechanically shape the variable loop's 2 diameter size without perforation of the wall of the insulative lumen 8, which could potentially interrupt or cross-connect with the high voltage carrying wires located inside the eccentric lumens 9. If these three properties are not selected and balanced appropriately, then the variable loop 2 will cease to function as intended. Without this protective non-conductive barrier, the end user is at an increased risk of electrical shock and the high voltage energy source and surrounding medical equipment are at risk of damage.

The pulsed field ablation catheter 1 of the present disclosure is developed around a proprietary platform including a steerable variable loop nitinol stylet mandrel. This steerable variable loop platform allows end users to size the loop diameter of the variable loop 2 to better interface with varying human anatomy. Without this feature, end users may require multiple catheters to complete a single electrophysiology procedure. Alternatively, an incorrect loop size may increase the difficulty to administer PFA therapy, which could result in increased time and cost to complete the procedure. The pulsed field ablation catheter 1 of the present disclosure leverages this proprietary platform on a therapeutic device. The pulsed field ablation catheter 1 can be sized with a shape-imparting mechanism 13 stylet by the end user to create pulsed field ablation lesions of varying size.

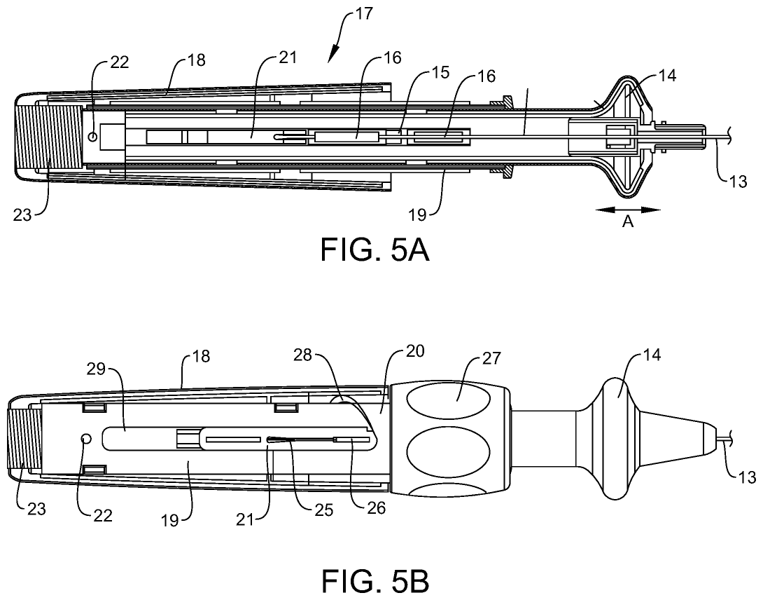

The pulsed field ablation catheter 1 of the first preferred embodiment includes a modified butt-joint 34 (see FIG. 8) and a modified tongue-and-groove joint 32. This allows composite tubes of different diameters to be thermally joined or welded. This is advantageous for this present embodiment for four main reasons, they are: 1) a smaller diameter loop 2 may be placed on the catheter shaft 3 for a more desirable tissue-electrode interface; 2) the larger diameter shaft 3 can transport diagnostic or interventional tools or devices 10 within a delivery lumen 6 without increasing its outer diameter to accommodate the smaller diameter loop 2, in addition to the tools or devices 10; 3) the different outer diameters can be designed more appropriately to enhance mechanical performance for steerability, pushability and kink resistance; and 4) the combined modified joints provide a stronger connection versus a single butt-joint used during conventional applications.

The tongue-and-groove joint 32 may be adapted to mate with a cooperatively shaped receiving portion mounted on the end of the shaft 3. In FIGS. 7 and 8A, the tongue has a generally square-shaped side profile. In FIG. 8B, the tongue has a generally rounded-shaped side profile. In FIG. 8C, the tongue has a generally pointed or trapezoidal-shaped side profile.

Preferably, the shaft is 11 French in diameter and this diameter may be referred to as a first diameter and the distal loop section is 7 French in diameter and this referred to a second diameter. Preferably, the first diameter is greater than the second diameter.

FIGS. 8A, 8B, and 8C depict potential design alternatives to the combined butt-joint and tongue-and-groove joint. In particular, the figures illustrate alternative designs of the tongue 33 in the modified tongue-and-groove joint 32. Preferably, the distal loop 31 includes a tongue portion 33 adapted to be mated with the corresponding end of the shaft 15 using the joint 34. The tongue may comprise a square shape 33a, a circular shape 33b, or a tapered design 33c. The tongue 33 may be thermally joined to the outer diameter of the deflection zone 5 where the thermal joint creates a groove 35 in which the tongue 33a-33c forms the tongue-and-groove joint 32. Positioned at a ninety-degree angle to the tongue 33 is the termination of the distal loop section 31 that forms the butt-joint 34. The two joints are modified to combine a bifurcation at which the variable loop 2 may depart from the deflection without occluding the delivery lumen 6.

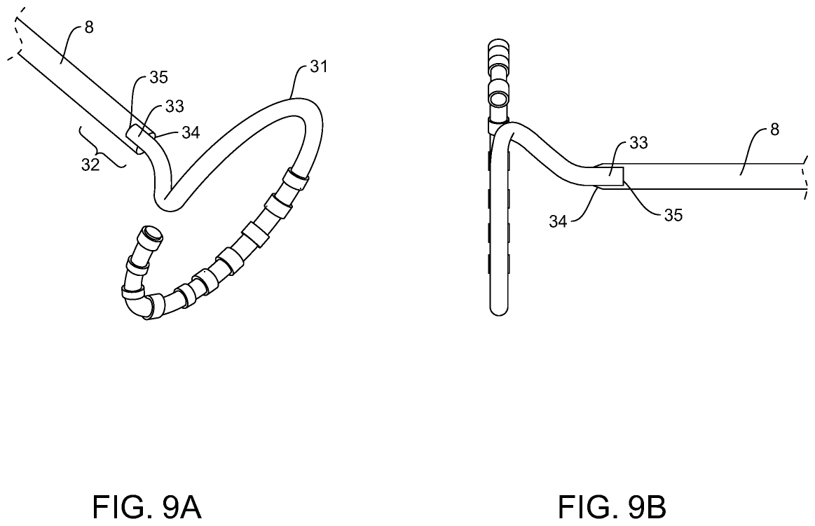

FIGS. 9A and 9B depict a profile and side view of a preferred design of the modified butt-joint and tongue-and-groove joint 32 between the variable loop 31 and the insulative lumen 8 of the multilumen composite catheter shaft 3 and deflection zone 5.

To further improve the mechanical characteristics of the variable loop 2, an insulative composite tube 31 is designed. This composite variable loop design includes a thin non-conductive braid fiber positioned at an angle to maximize flexibility and torsion. The variable loop composite tube 31 is further designed to improve kink resistance so that the variable loop's 2 smaller diameter sizes do not result in unsightly kinking of the composite tube 31 that may also contribute to thrombus formation and stroke. Design of this non-conductive composite tube 31 for mechanical performance and kink resistance requires careful selection of polymer materials based on their flexural modulus, Poisson's ratio, shear modulus, and inner and outer radii.

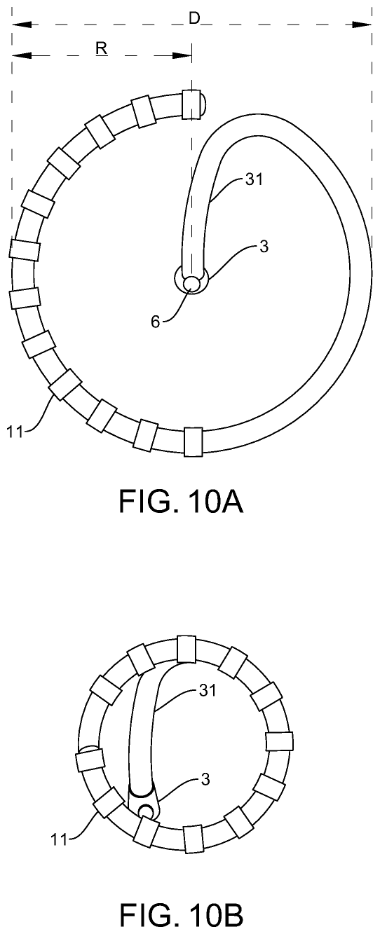

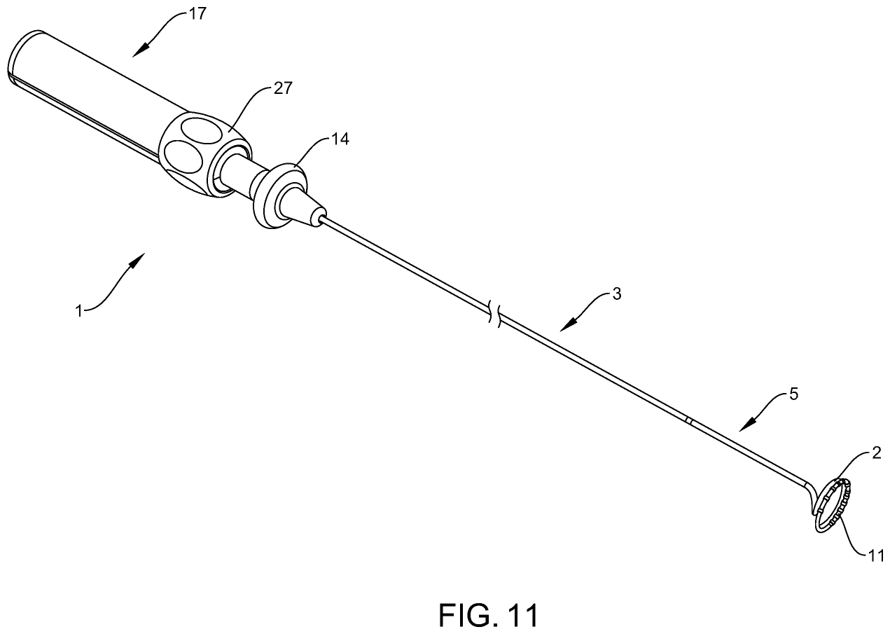

FIGS. 10A and 10B depict a preferred embodiment of the catheter's distal variable loop 31 illustrating a large diameter loop 2a and a small diameter loop 2b wherein the high-voltage delivering electrodes 11 do not cross one another during loop sizing. FIGS. 10A and 10B also depict the variable loop 31 in two different configurations. Preferably, the maximum extended variable loop 2 is depicted in FIG. 10A wherein the loop 31 is deflected into a large loop configuration by the catheter handle actuation. FIG. 10A depicts the large loop extending in a circle or lasso configuration in clockwise direction with twelve ablation electrodes 11 equidistantly spaced about the second half of the circle. In this large or extended configuration, the end of the loop 31 forms a circle without touching or overlapping the remainder of the circle or the lower parts of the loop 31.

Preferably, the maximum circle size is determined by the circle being formed but the loop 31 doesn't touch or contact itself when viewed from the top view depicted in FIG. 10A.

FIG. 10B depicts the loop 31 wherein the diameter and radii of the circle have been reduced in size by actuation of the catheter handle. The reduction in diameter generally causes the tail or end of the loop 31 to overlap with itself or the circle. The lack of overlapping electrodes significantly reduces the risk of electrical shorting across the electrodes, particularly wherein the overlapping portion touches or presses against the circle.

Preferably, the ablation electrodes are positioned or mounted on the later or second half of the circumference, which is equal to πR of the circle in FIG. 10A. In FIG. 10B, the circle is reduced in size to 1/2D or half diameter of the circle in FIG. 10A. The end of loop 31 overlaps and/or contacts the remainder or lower parts of the loop 31. However, the ablation electrodes 11 in FIG. 10B form a complete loop without the electrodes overlapping with the possibility of electrical shorting. The portion of the loop with the electrodes is only overlapping the portion of the loop 31 with electrodes mounted on it.

Preferably, the plurality of electrodes is adapted to be mounted or affixed to the arc of the second half of the circle in the largest sizing configuration of the steering loop. When the sizing of the steering loop is minimized, this arc significantly overlaps the other parts of the steering loop. However, the other portions of the steering loop or circle not forming part of the arc do not include electrodes and therefore this feature prevents or limits electrical shorting.

Preferably, a steerable loop wherein the loop forms a circle transverse to the longitudinal direction of the shaft wherein the diameter of the circle may be amended between a minimum diameter and maximum diameter by a steering mechanism.

The catheter 1 of the present disclosure delivers voltages greater than 300 volts. The voltage passes through the catheter shaft through small (OD) insulated wires. The wires pass parallel to the nitinol shape-imparting mechanism 13 and they terminate at the plurality of electrodes 11 arranged on the distal-most variable loop 2. Each wire corresponds to a single electrode. In some embodiments, the electrodes are arranged in pairs otherwise known as channels. Each electrode pair is synchronized to deliver either bipolar monophasic waveforms or bipolar biphasic waveforms. The synchronized pulse delivery works by one electrode 11 in each channel emitting a voltage and a current. That voltage and current then passes through the resistive heart tissue and exits through the other electrode 11 of the pair. Because high voltages greater than 300 volts are expected, the composite tube 15 of loop 2 under the electrodes 11 is made of an insulative material. Preferably, the shaft of the catheter is constructed of a silicone polymer.

Additionally, further embodiments may include an additional feature (not shown) wherein the shaft includes metallic or metallized braid up to the start of the deflection zone of the catheter, and extending beyond the start the of deflection zone, the braid is constructed of a non-metallic braid up to the distal end. Preferably, the region underneath or close to the electrodes may be non-metallic braid.

Design of braided composite tubes for a catheter is not trivial. The preferred embodiments of the present disclosure are based on Classical Laminate Theory that calculates the optimized braid dimensions, braid angle, and braid coverage for superior mechanical performance. The theory begins by predicting the stiffness of the material through the Rule of Mixtures theorem (1). It then progresses to Classical Laminate Analysis where a precise stiffness can be obtained.

E_{c}=E_{f}V_{f}+EₘVₘ (1)

The Rule of Mixtures formula (1) predicts the stiffness of a composite material. It can be used to calculate the Young's Modulus, density, Poisson's ratio, and strength of the composite laminate. The Rule of Mixtures for Young's Modulus assumes uni-directional fibers and predicts the Young's Modulus only in the direction of the fiber. To predict the Young's Modulus of a composite tube according to a fiber angle, the more advanced Classical Laminate Analysis is employed.

For Classical Laminate Analysis, four elastic constants are needed to characterize the in-plane macroscopic elastic properties of the composite. First, the longitudinal stiffness using the Rule of Mixtures Formulae (2) is derived. Then, the transverse stiffness using the Inverse Rule of Mixtures Formulae (3) is used. The Major Poisson's ratio using the Rule of Mixtures for Poisson's ratio (4) is calculated. Lastly, the in-plane shear modulus is derived using the Inverse Rule of Mixtures for Shear equation (5). Once the above properties are established, then the four material elastic properties can be expressed, which is easily done in matrix form.

$${E}_{c} = {E}_{f} {V}_{f} + {E}_{m} ( 1 - {V}_{f} ) \qquad ( 2 )$$

$$\frac{1}{{E}_{c}} = \frac{{V}_{f}}{{E}_{f}} + \frac{{V}_{m}}{{E}_{m}} \qquad ( 3 )$$

$${v}_{12} = {v}_{f} {V}_{f} + {v}_{m} ( 1 - {V}_{f} ) \qquad ( 4 )$$

$$\frac{1}{{G}_{12}} = \frac{{V}_{f}}{{G}_{f}} + \frac{( 1 - {V}_{f} )}{{G}_{m}} \qquad ( 5 )$$

There are two matrices needed for Classical Laminate Analysis. The first being the Stiffness Matrix (6) and the second being the Compliance Matrix (7), which is the inverse of Stiffness Matrix (6). The Stiffness Matrix (6) calculates laminate stresses from laminate strains whereas the Compliance Matrix (7) calculates laminate strains from laminate stresses. A Transformation Matrix (8) is needed to transform the Stiffness (9) and Compliance (10) components. Individual Compliance and Stiffness terms are derived from the Transformation Matrix with a focus on individual stiffness terms. From these terms, the composite's Elastic and Shear Moduli can be derived to solve for flexural (11), axial (12), and torsional (13) rigidity.

$$\{ \begin{matrix}{σ}_{x} \\ {σ}_{y} \\ {τ}_{xy}\end{matrix} \} = [ \begin{matrix}{\overline{Q}}_{11} & {\overline{Q}}_{12} & {\overline{Q}}_{16} \\ {\overline{Q}}_{21} & {\overline{Q}}_{22} & {\overline{Q}}_{26} \\ {\overline{Q}}_{16} & {\overline{Q}}_{26} & {\overline{Q}}_{66}\end{matrix} ] \{ \begin{matrix}{ε}_{x} \\ {ε}_{y} \\ {γ}_{xy}\end{matrix} \} \qquad ( 6 )$$

$$\{ \begin{matrix}{ε}_{x} \\ {ε}_{y} \\ {γ}_{xy}\end{matrix} \} = [ \begin{matrix}{\overline{S}}_{11} & {\overline{S}}_{12} & {\overline{S}}_{16} \\ {\overline{S}}_{21} & {\overline{S}}_{22} & {\overline{S}}_{26} \\ {\overline{S}}_{16} & {\overline{S}}_{26} & {\overline{S}}_{66}\end{matrix} ] \{ \begin{matrix}{σ}_{x} \\ {σ}_{y} \\ {τ}_{xy}\end{matrix} \} \qquad ( 7 )$$

$$\{ \begin{matrix}{σ}_{L} \\ {σ}_{T} \\ {τ}_{LT}\end{matrix} \} = [ \begin{matrix}{cos}^{2} θ & {sin}^{2} θ & 2 sin θcos θ \\ {sin}^{2} θ & {cos}^{2} θ & - 2 sin θ cos θ \\ - sin θ cos θ & sin θcos θ & {cos}^{2} - {sin}^{2} θ\end{matrix} ] \{ \begin{matrix}{σ}_{x} \\ {σ}_{y} \\ {τ}_{xy}\end{matrix} \} \qquad ( 8 )$$

$${\overline{Q}}_{11} = {Q}_{11} {cos}^{4} θ + {Q}_{22} {sin}^{4} θ + ( 2 {Q}_{12} + {Q}_{66} ) {cos}^{2} θ {sin}^{2} θ \\ {\overline{Q}}_{12} = ( {Q}_{11} + {Q}_{22} - 4 {Q}_{66} ) {cos}^{2} θ {sin}^{2} θ + {Q}_{12} ( {cos}^{4} θ + {sin}^{4} θ ) \\ {\overline{Q}}_{22} = {Q}_{11} {sin}^{4} θ + {Q}_{22} {cos}^{4} θ + ( 2 {Q}_{12} + 4 {Q}_{66} ) {cos}^{2} θ {sin}^{2} θ \\ {\overline{Q}}_{66} = ( {Q}_{11} + {Q}_{22} - 2 {Q}_{12} - 2 {Q}_{66} ) {cos}^{2} θ {sin}^{2} θ + {Q}_{66} ( {cos}^{4} θ + {sin}^{4} θ ) \qquad ( 9 )$$

$${\overline{Q}}_{16} = ( {Q}_{11} - 2 {Q}_{66} - {Q}_{12} ) {cos}^{3} θ {sin}^{3} θ - ( {Q}_{22} - {Q}_{12} - 2 {Q}_{66} ) cos θ {sin}^{3} θ \\ {\overline{Q}}_{26} = ( {Q}_{11} - 2 {Q}_{66} - {Q}_{12} ) cos θ {sin}^{3} θ - ( {Q}_{22} - {Q}_{12} - 2 {Q}_{66} ) {cos}^{3} θ sin θ$$

$${\overline{S}}_{11} = {S}_{11} {cos}^{4} θ + {S}_{22} {sin}^{4} θ + ( 2 {S}_{12} + {S}_{66} ) {cos}^{2} θ {sin}^{2} θ \\ {\overline{S}}_{12} = ( {S}_{11} + {S}_{22} - {S}_{66} ) {cos}^{2} θ {sin}^{2} θ + {S}_{12} ( {cos}^{4} θ + {sin}^{4} θ ) \\ {\overline{S}}_{22} = {S}_{11} {sin}^{4} θ + {S}_{22} {cos}^{4} θ + ( 2 {S}_{12} + {S}_{66} ) {cos}^{2} θ {sin}^{2} θ \\ {\overline{S}}_{66} = 4 ( {S}_{11} + {S}_{12} - 2 {S}_{12} ) {cos}^{2} θ {sin}^{2} θ + {{S}_{66} ( {cos}^{2} θ - {sin}^{2} θ )}^{2} \qquad ( 10 )$$

$${\overline{S}}_{16} = ( 2 {S}_{11} - {S}_{66} - 2 {S}_{12} ) {cos}^{3} θ sin θ - ( 2 {S}_{22} - 2 {S}_{12} - {S}_{66} ) cos θ {sin}^{3} θ \\ {\overline{S}}_{26} = ( 2 {S}_{11} - {S}_{66} - 2 {S}_{12} ) cos θ {sin}^{3} θ - ( 2 {S}_{22} - 2 {S}_{12} - {S}_{66} ) {cos}^{3} θ sin θ \\ {S}_{11} = \frac{1}{{E}_{L}} , {S}_{12} = - \frac{{μ}_{LT}}{{E}_{L}} = - \frac{{μ}_{TL}}{{E}_{T}} , {S}_{22} = \frac{1}{{E}_{T}} , {S}_{66} = \frac{1}{{G}_{LT}} .$$

$${Q}_{11} = \frac{{E}_{L}}{( 1 - {μ}_{LT} {μ}_{TL} )} , {Q}_{12} = \frac{{μ}_{LT} {E}_{T}}{( 1 - {μ}_{LT} {μ}_{TL} )} = \frac{{μ}_{TL} {E}_{L}}{( 1 - {μ}_{LT} {μ}_{TL} )} , \qquad ( 11 )$$

$${Q}_{22} = \frac{{E}_{T}}{( 1 - {μ}_{LT} {μ}_{TL} )} , {Q}_{66} = {G}_{LT} \qquad ( 12 )$$

$$EI = {E}_{xx} \frac{π ( {r}_{0}^{4} - {r}_{i}^{4} )}{4} \qquad ( 13 )$$

$$EA = {E}_{xx} π ( {r}_{0}^{2} - {r}_{i}^{2} ) \qquad ( 14 )$$

$$GJ = {G}_{xy} \frac{π ( {r}_{0}^{4} - {r}_{i}^{4} )}{2} \qquad ( 15 )$$

Because the catheter loop 2 is variable, the catheter tube is designed such that the tube does not kink or become occluded during loop sizing. A kink or occlusion of the catheter tube is both unsightly and it may become a thrombus trap and/or may cause jamming in hemostatic introducers. It could also damage the internal catheter parts such as the high-voltage carrying wires. To address this problem, the catheter tube's critical radius is estimated using the Brazier Effect (16) and Classical Structural Mechanics formulae (17). Because buckling (or kink) phenomena is unstable and therefore difficult to predict precisely, an upper and lower bound is determined to better understand the critical radius. To do this, first the tangent modulus (18) is calculated and then one can evaluate the reduced modulus (19) or lower bound as a function of the Young's and tangent moduli. The critical inelastic buckling load can then be determined using Euler's Buckling Equations for Simply Supported Columns (20) while evaluating the tangent and reduced (19) moduli individually.

$$\overline{M} = \frac{2 \sqrt{2}}{9} \frac{E π {rt}^{2}}{\sqrt{1 - {v}^{2}}} \qquad ( 16 )$$

$$a + δ \geq {r}_{critical} = \frac{\overline{M}}{{P}_{c}} \qquad ( 17 )$$

$$\frac{{E}_{t}}{E} = \frac{1}{1 + \frac{3}{7} {n ( \frac{σ}{{σ}_{0.7}} )}^{n - 1}} ; n = 1 + \frac{ln ( \frac{17}{7} )}{ln ( \frac{{σ}_{0.7}}{{σ}_{0.85}} )} \qquad ( 18 )$$

$${E}_{r} = \frac{2 {EE}_{t}}{E + {E}_{t}} \qquad ( 19 )$$

$${P}_{c} = {π}^{2} {E}_{r} \frac{1}{{L}^{2}} \qquad ( 20 )$$

To maintain the catheter's steerable function, the deflection zone 5 is designed to have a relatively small bending stiffness while maximizing torsional rigidity. The relatively small bending stiffness is needed to mitigate the bending stiffness of certain diagnostic and interventional tools and devices 10. This way the catheter maintains its steer-ability even during the use of the tools or devices. For example, as a tool or device 10 is passed through the catheter's delivery lumen 6 the combined bending stiffness of the tool or device 10 as well as the catheter deflection zone 5 is relatively small so that the catheter's internal steering mechanism 13 can mechanically deflect the catheter and the tool or device 10. The combined bending stiffness must not only be relatively small to allow proper function of the shape-imparting mechanism 13, but it must have a low bending stiffness so the end user can apply a relatively low actuation force on the handle's plunger 14 to mechanically deflect the deflection zone 5 of the catheter.

The design of the catheter's deflection zone 5 first considers the bending stiffness of potential diagnostic and interventional tools or devices 10. The bending stiffness may be measured and calculated using the flexural rigidity (21) equation. For further mechanical analysis, axial and torsional rigidity may be measured and calculated using equations (22) and (23). Once the diagnostic or interventional tool's 10 flexural rigidity is obtained, then the bending stiffness of the multilumen extrusion may be calculated by way of Finite Element Analysis. Finally, the braid mechanics may be designed and determined using Classical Laminate Theory analysis (1-15).

$$EI = \frac{{PL}^{3}}{δ} \qquad ( 21 )$$

$$EA = \frac{PL}{δ} \qquad ( 22 )$$

$$GJ = \frac{TL}{φ} \qquad ( 23 )$$

Because the multilumen extrusion has an irregular profile, Finite Element Analysis (FEA) is needed to determine flexural, axial, and torsional rigidity. Applying the appropriate loading and boundary conditions, FEA produces a displacement plot. Max displacements in the appropriate axis may be used in equations (21-22) for post processing of flexural and axial rigidity values. However, the angle of twist φ requires additional post processing as most FEA programs only provide one-dimensional displacements. The angle of twist φ may then be obtained by applying equations (24). With FEA the right material may be selected such that the bending stiffness is relatively low, the torsional rigidity is relatively high, and the ultimate tensile strength is relatively high. This design approach permits the catheter's steerable function while also reducing the potential for the multilumen extrusion to rupture or tear.

$$φ = {cos}^{- 1} ( \frac{{a}^{2} + {b}^{2} - {c}^{2}}{2 ab} ) ; c = \sqrt{{x}^{2} + {z}^{2}} ; a = b = r \qquad ( 24 )$$

The multilumen and braid fiber material may be optimized to achieve the desired bending stiffness in the catheter to maintain steerability during the use of diagnostic or interventional tools or devices through the catheter's delivery lumen 6. The materials may comprise a mixture of materials to achieve the desired bending stiffness. The mixture's material properties may be further analyzed by way of the Rule of Mixture theorem (1). The combined bending stiffness of the braided fiber tube 12, multilumen extrusion, and diagnostic or interventional tools and devices 10 shall be relatively low so the end user may apply a reduced actuation force on the catheter's plunger 14 located on the catheter's handle.

The reduced actuation force applied by the user and administered onto the handle's plunger mechanism 14 is defined by equation (25) where the bending moment M is used to deflect the catheter assembly using the reduced actuation force F. The nonlinear beam deflection formulae (26 & 27) may then be used to evaluate a desired deflection of the loop to about 180° given the calculated bending stiffness as previously defined.

$$M = {F}_{actuation} r \qquad ( 25 )$$

$$y ( x ) = - \frac{EI \sqrt{1 - {( \frac{M}{EI} )}^{2} {x}^{2}}}{M} + \frac{EI}{M} \qquad ( 26 )$$

$$S = \frac{\sqrt{\frac{{( EI )}^{2}}{{( EI )}^{2} - {M}^{2} {x}^{2}} \sqrt{{( EI )}^{2} - {M}^{2} {x}^{2}} {tan}^{- 1} ( \frac{Mx}{\sqrt{{( EI )}^{2} - {M}^{2} {x}^{2}}} )}}{M} \qquad ( 27 )$$

Further design of the catheter's multilumen extrusion includes the low-flow, low-pressure characteristics of the delivery lumen 6. Although low pressures generally occur during low flow conditions, flow in an annulus is particularly challenging to achieve low pressure profiles. The annulus is created when an interventional or diagnostic tool or device 10 is introduced inside the lumen. As a result, flow in a microlumen annulus, as described herein, further compounds the problem of maintaining low pressures. Therefore, the design leverages Fluid Dynamics theory to maintain the smallest lumen profile while also keeping the pressure drop through the lumen below pressure requirements for a standard intravenous pressure bag.

The delivery lumen 6 is designed for two use scenarios: firstly, the flow vs pressure profiles are designed for a simple concentric delivery lumen 6 and secondly, the flow vs pressure profiles are designed for fluid flow in an annulus such that low-flow low-pressure conditions occur during use of interventional or diagnostic tools or devices 10 to reduce the risk of thrombus formation. In both use case scenarios, the governing equations change quite drastically.

In the first use case scenario, the delivery lumen 6 is designed like a simple unobstructed concentric tube. First the velocity of fluid (28) must be determined according to the flow rate and the area of fluid profile. Equation (29) is used to determine the Reynold's number, which is a function of the fluid properties and velocity. Finally, the pressure drop (30) may be determined for by calculating the major and minor losses in the system.

$$\dot{V} = {A}_{f} V \qquad ( 28 )$$

$$Re = \frac{internal forces}{viscous forces} = \frac{ρ {D}_{h} {V}_{p}}{μ} = \frac{{D}_{h} V}{v} \qquad ( 29 )$$

$$Δ P = {[ \frac{fL}{{D}_{h}} \frac{1}{2} ρ {V}^{2} ]}_{major losses} + {[ {\sum }_{i = 1}^{n} {k}_{i} \frac{1}{2} ρ {V}^{2} ]}_{minor losses} \qquad ( 30 )$$

For the second use case scenario, the delivery lumen 6 is designed to be partially obstructed by a diagnostic or interventional tool or device. This use case scenario is otherwise known as a flow in an annulus. This condition is particularly challenging to achieve low-flow low-pressure outcomes. Because the tool or device partially obstructs the flow of the fluid, the inner diameter of delivery lumen 6 must be increased to compensate for such an obstruction. However, increasing the delivery lumen inner diameter may increase the outer diameter of the multilumen composite catheter shaft 3 too much and the pulsed field ablation catheter 1 becomes impractical for clinical use. Additionally, decreasing the inner diameter of the delivery lumen 6 to improve practicality may result in a pressure drop that requires mechanical pumps to compensate for the pressure drop. Therefore, the delivery lumen inner diameter should be sized for low-flow low-pressures with the use of pressure bags to push the fluid over the annulus. This makes the device practical both from a use case and cost saving. To achieve the desired fluid dynamics for flow over an annulus, first the hydraulic diameter (31) is calculated, then the velocity of fluid, and finally the pressure drop (33) is used to determine the low pressure for a low fluid flow catheter.

$${D}_{h} = \frac{4 π ( {r}_{o}^{2} - {r}_{i}^{2} )}{2 π ( {r}_{o} + {r}_{i} )} = 2 ( {r}_{o} - {r}_{i} ) \qquad ( 31 )$$

$$V = \frac{Q}{π ( {r}_{o}^{2} - {r}_{i}^{2} )} \qquad ( 32 )$$

$$Δ P = {\frac{8 μ lQ}{π} [ {r}_{o}^{4} - {r}_{i}^{4} - \frac{{( {r}_{o}^{2} - {r}_{i}^{2} )}^{2}}{ln ( \frac{{r}_{o}}{{r}_{i}} )} ]}^{- 1} \qquad ( 33 )$$

Clinically, the variable loop pulsed field ablation catheter 1 is advantageous for three reasons, they are: 1) the distal loop is variable, 2) the source of ablation is non-thermal and tissue selective, and 3) a thrombus entrapment point is eliminated by the use of a continuous fluid flow or lavage.

The catheter's variable loop 2 allows end users to create a variety of circular loop diameters without needing to use additional catheters during a single procedure. This allows for greater flexibility and efficiency of use during medical procedures, further reducing the economic burden on healthcare payers. The plurality of ablation electrodes 11 are positioned on the distal most portion of the variable loop 2. This feature eliminates events where the electrodes can cross or touch that may lead to electrical shorting, surging and/or arcing. This is important because it protects end users from electrical shock or device related injury.

The catheter as an interventional pulsed field ablation (PFA) catheter is advantageous because PFA is a non-thermal and a tissue selective ablation modality. This adds to the catheter's design complexity. PFA requires high voltages and currents (e.g., >300 V and >1 amp). The high-voltage carrying wires must be thoroughly insulated and protected from damage. In particular, the mechanical deflection of the shape-imparting steering mechanism 13, by the proprietary deflectable nitinol mandrel system, must not damage the high-voltage carrying wires during mechanical motion. This potential outcome is mitigated by an insulative lumen 8 that separates the shape-imparting steering mechanism 13 from the high-voltage carrying wires. The insulative lumen 8 is further separated and electrically isolated from the delivery lumen 6 by the septal wall 7. This feature permits a bifurcation to occur at the distal deflection zone 5 and the proximal end of the variable loop 2.

The bifurcation that occurs between the delivery lumen 6 and the insulative lumen 8 is further joined by a modified butt-joint and tongue-and-groove joint 32. This permits the variable loop 2 to be thermally joined to the catheter's deflection zone 5 having a larger outer diameter than the proximal end of the variable loop 2. Not only does the modified butt-joint and tongue-and-groove joint 32 assist the joining of two different diameter tubes but it also improves the strength of the joint overall.

The catheter's delivery lumen 6 acts as a route through which interventional tools or devices 10 may be guided and introduced inside the cardia is particularly useful as these tools or devices may be used for better positioning and stability of the catheter in addition to diagnostic sensing of ECG signals. Unfortunately, this route and distal aperture 6 may act as a thrombus trap further contributing to the risk of procedure related stroke. This risk is mitigated by a low-flow low-pressure fluid flow that provides continuous irrigation or lavage, such that surrounding blood does become entrapped in the lumen and forms a thrombus, which may result in stroke. Additionally, the low-flow low-pressure delivery lumen 6 performance eliminates the need for costly pumps required to overcome a large pressure drop created by the flow in an annulus. As a result, the low-flow low-pressure fluid flow permits the use of pressurized saline bags that are available in almost any hospital further reducing the cost and complexity of the procedure while making the device available to under resourced nations.

Regarding the disadvantages of prior art, the variable loop PFA catheter 1 is a steerable catheter that can adapt to various intracardiac landscapes. This is particularly helpful in a global market where patient anatomies change regionally. The catheter achieves such outcomes through precise mechanical design of the catheter's deflection zone 5 by incorporating Classical Laminate Theory, Finite Element Analysis, Brazier Effect, Classical Structural Mechanics, and Fluid Dynamics. As such, the present disclosure overcomes or ameliorates at least one of the disadvantageous of the prior art by innovative mechanical design and performance.

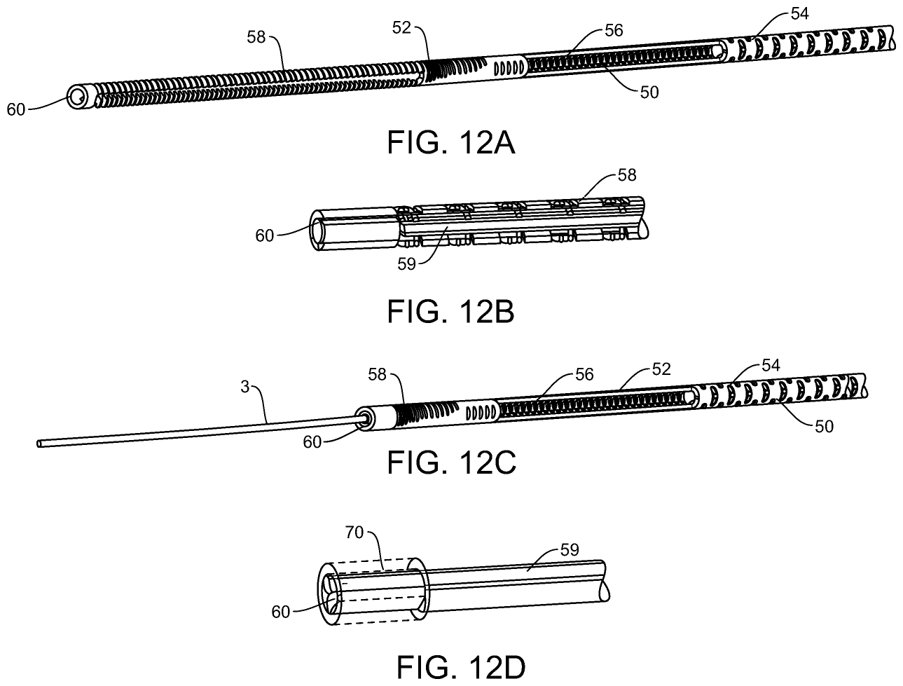

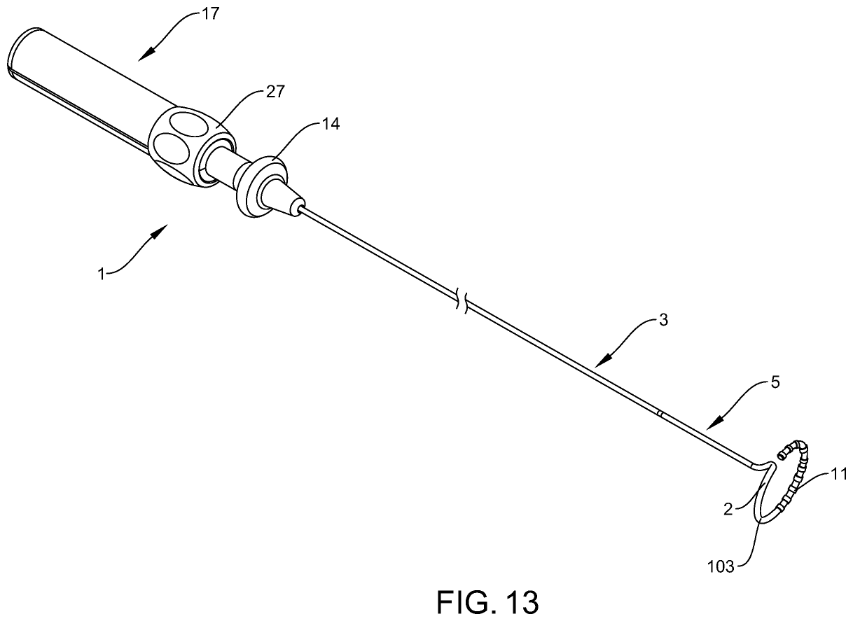

FIGS. 12A-12D show an alternative exemplary embodiment of a steering mechanism for use in any of the embodiments of the catheter of the present disclosure. FIG. 12A shows a distal end portion 52 of a catheter 50. Distal end portion 52 includes, from proximal to distal, a catheter body 54, a deflection zone 56, and a steering tube 58. FIG. 12B shows a pull wire 59 that extends through catheter body 54 and deflection zone 56 and that is connected to shaft 3. As shown in FIG. 12C, shaft 3 extends distally from a distal tip 60 and connects to loop 2, shown in FIG. 13. When pull wire 59 is pulled proximally, loop 2 contracts.

FIG. 12D shows an exemplary method for attaching pull wire 59 to distal tip 60. A crimp tube 70 is placed over distal tip 60 and then the distal end of pull wire 59 is laser welded to distal tip 60.

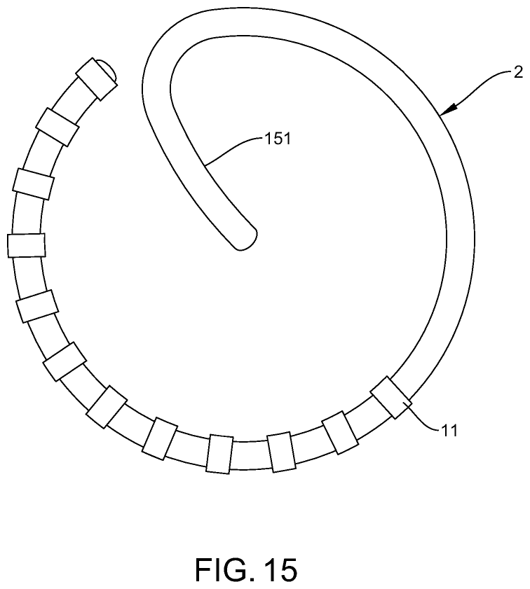

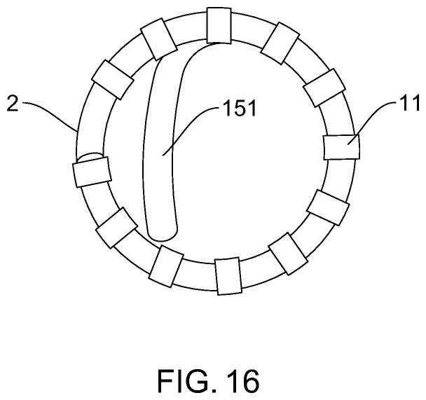

A further preferred embodiment is depicted in FIGS. 13 to 18. In this embodiment, there is presented a variable loop ablation catheter for cardiac ablation treatment. A similar numbering pattern has been applied to these figures for reference. Preferably, the distal loop 2 is of variable diameter. The diameter is controlled by the corresponding steering mechanism positioned in the handle, along with the steering tube 58 and pull wire 59 discussed above. Preferably, the loop on the distal end is configured to be transverse or at 90 degrees relative to the elongated shaft of the catheter. The loop may be configured form a single loop or circle at its maximum diameter.

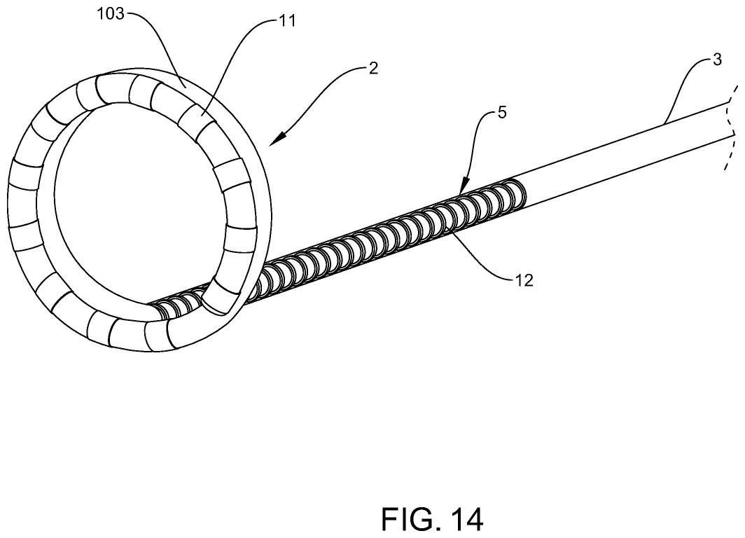

In FIG. 14, the variable loop catheter includes a single lumen shaft 3 and the catheter includes a first and only steerable loop 103. This loop 103 serves a similar function to loop 100 in the earlier described embodiments.

Wherein the diameter is reduced, the loop overlaps itself in a tight helix, which is sometimes called a pig-tail style loop. As per some of the previously described embodiments, the present embodiment includes electrodes mounted on half of the circumference of the circle formed by the maximum diameter. The maximum diameter loop is depicted in FIGS. 13, 15 and 18. The compact configuration of the loop with the reduced diameter is depicted in FIGS. 12, 14, 16 and 17. One of advantages of the feature of limiting the placement of the electrodes is that when the loop is in a compact configuration the electrodes from different parts or portions of the helix do not overlap and thereby reduces the likelihood of electrical shortening across the electrodes when in use.

Further, FIGS. 12, 13, 15, 16 depict a catheter configuration wherein the shaft is positioned centrally in respect of the circle or loop when viewed from the top view of the distal end. This configuration includes a small positioning arm 151 extending from the shaft and joining with the transverse loop 2. The positioning arm 151 allows the shaft is positioned within the circle formed by the loop when in use. In some situations, this may be of surgical advantage wherein the clinician is required to rotate the positioning of the loop to full maximal ablation coverage, when in use.

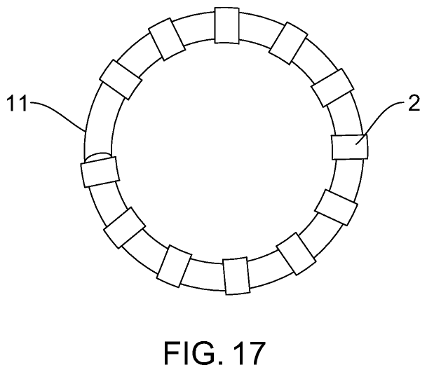

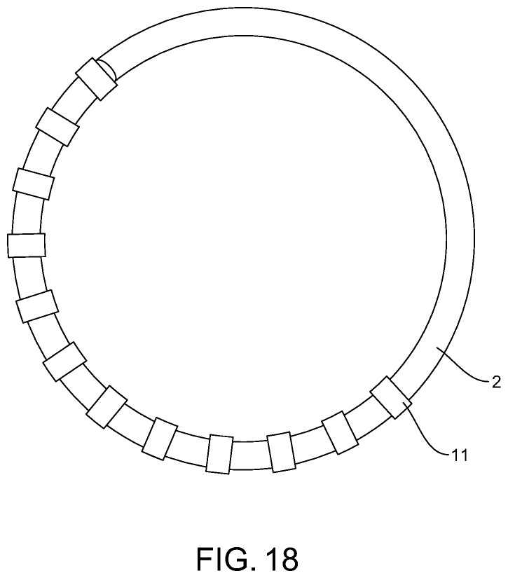

FIGS. 17 and 18 depict a slight variant embodiment, wherein the shaft is positioned directly underneath the circumference of the circle forming the loop 2 when viewed from the top view of the distal end. The shaft in this configuration is position in direct alignment with the circumference making the loop eccentric (e.g., not mounted on the axis of the circle). The advantage with this configuration is that it may allow clinician to apply additional pressure through the loop of the catheter against the inner walls of the region that is being treated with ablation therapy.

Although the present disclosure has been described with reference to specific examples, it will be appreciated by those skilled in the art that the present disclosure may be embodied in many other forms, in keeping with the broad principles and the spirit of the present disclosure described herein.

The present disclosure and the described preferred embodiments specifically include at least one feature that is industrial applicable.

1.-21. (canceled)

22. A cardiac ablation catheter comprising:
i. a shaft having a proximal end, a distal end, and at least one insulative lumen and at least one delivery lumen;
ii. a first steerable loop capable of being inserted into the insulative lumen and a second steerable loop capable of being inserted into the delivery lumen and wherein the delivery lumen is adapted to mounted within the insulative lumen;
iii. a handle coupled to the proximal end of the shaft, the handle having a steering mechanism;
iv. a shape imparting element having a proximal end and a distal end; the proximal end is adapted to be coupled to the steering mechanism and the distal end of the element is adapted to be coupled to the steerable loop and wherein the shape imparting element is positioned within the insulative lumen;
v. a plurality of electrodes positioned on the steerable loop electrically coupled to at least one electrical connector, the at least one electrical connector being configured to electrically coupled with an electrical pulsed field ablation energy source to power the plurality of electrodes for treatment; and
vi. wherein the first steering loop is reduced in size from a maximum circle to a minimum circle, wherein the minimum circle is half the diameter of the maximum circle, wherein the plurality of the electrodes is positioned apart around the circumference of the circle.

23. The catheter of claim 22, wherein the shaft has a first diameter, a proximal end and a distal end, wherein the distal end is joined to a distal loop section of the first steerable loop having a second diameter, and wherein the join is achieved by the mating of a tongue and groove positioned on corresponding mating portions of the distal loop section and the distal end.

24. The catheter of claim 23, wherein the catheter includes a plurality of lumens.

25. The catheter of claim 22, further comprising a plurality of electrodes positioned on the second steerable loop, the electrodes being configured to be electrically coupled with an electrical pulsed field ablation energy source to power the plurality of electrodes for treatment; and wherein the plurality of electrodes are spaced at predetermined intervals on an arc of the second half of the circle.

26. The catheter of claim 25, wherein the predetermined intervals are equidistant.

27. The catheter of claim 26, wherein the plurality of electrodes is twelve electrodes.

28. The catheter of claim 27, wherein the electrodes comprise alternative positive and negative electrodes.

29. The catheter of claim 28, wherein adjacent pairs of electrodes are configured to fire simultaneously.

30. The catheter of claim 28, wherein localized first pairs of similar electrodes are configured to fire simultaneously, with corresponding localized second pairs of opposite electrodes configured to fire subsequent to the first pairs.

31. The catheter of claim 27, wherein the steerable loop in a configuration between the maximum and minimum diameters has no electrode overlap other electrodes on the steerable loop.

32. The catheter of claim 22, further comprising a non-metallic braid encapsulated within the wall of the shaft proximal to the distal end of the shaft.

33. The catheter of claim 32, wherein the braid is constructed of a non-electrically conductive material.

34. A cardiac ablation catheter comprising:
i. a shaft having a proximal end, a distal end, and at least one insulative lumen;
ii. a first steerable loop capable of being inserted into the insulative lumen;
iii. a handle coupled to the proximal end of the shaft, the handle having a steering mechanism;
iv. a shape imparting element having a proximal end and a distal end; the proximal end is adapted to be coupled to the steering mechanism and the distal end of the element is adapted to be coupled to the steerable loop and wherein the shape imparting element is positioned within the insulative lumen;
v. a plurality of electrodes positioned on the steerable loop electrically coupled to at least one electrical connector, the at least one electrical connector being configured to electrically coupled with an electrical pulsed field ablation energy source to power the plurality of electrodes for treatment;
vi. wherein a plurality of electrodes positioned on the first steerable loop, the electrodes are configured to be electrically coupled with an electrical pulsed field ablation energy source to power the plurality of electrodes for treatment; and wherein the plurality of electrodes are spaced at predetermined intervals on the arc of the second half of the circle; and
vii. wherein the first steering loop is reduced in size from a maximum circle to a minimum circle, wherein the minimum circle is half the diameter of the maximum circle, wherein the plurality of the electrodes is positioned apart around the circumference of the circle.

35. The catheter of claim 34, wherein the shaft has a first diameter, a proximal end and a distal end, and wherein the distal end is joined to a distal loop section having a second diameter, and wherein the join is achieved by the mating of a tongue and groove positioned on corresponding mating portions of the distal loop section and the distal end.

36. The catheter of claim 35, wherein the plurality of electrodes is positioned around the circumference of the circle when the steerable loop is reduced to the minimum circle.

37. The catheter of claim 36, wherein the predetermined intervals are equidistant.

38. The catheter of claim 37, wherein the plurality of electrodes is twelve electrodes.

39. The catheter of claim 38, wherein the steerable loop in a configuration between the maximum and minimum diameters has no electrode overlap other electrodes on the steerable loop.

40. The catheter of claim 39, further comprising a non-metallic braid encapsulated within the wall of the shaft proximal to the distal end of the shaft.

41. The catheter of claim 40, wherein the braid is constructed of a non-electrically conductive material.
